(19) United States
(12) Patent Application Publication
(10) Pub. No.: US 2026/0066111 A1
(43) Pub. Date: Mar. 5, 2026

(54) MACHINE LEARNING BASED NON-INVASIVE ASSESSMENT OF CORONARY MICROVASCULAR DISEASE FROM PCCT IMAGES

(71) Applicant: Siemens Healthineers AG, Forchheim (DE)

(72) Inventors: Lucian Mihai Itu, Brasov (RO); Puneet Sharma, Princeton Junction, NJ (US)

(21) Appl. No.: 18/817,287

(22) Filed: Aug. 28, 2024

(51) Int. Cl. G16H 50/20; G16H 10/60; G16H 50/50

(52) U.S. Cl. CPC G16H 50/20; G16H 10/60; G16H 50/50

(57) ABSTRACT

Systems and methods for determining an assessment of coronary microvascular disease of the patient are provided. Patient data of a patient is received. The patient data comprises patient characteristics and vessel characteristics. An assessment of coronary microvascular disease of the patient is determined based on the patient data using a machine learning based assessment model. Results of the assessment of coronary microvascular disease of the patient are output.

### TECHNICAL FIELD

The present invention relates generally to machine learning based assessment of coronary microvascular disease, and in particular to machine learning based non-invasive assessment of coronary microvascular disease from PCCT (photon counting computed tomography) images.

### BACKGROUND

CMD (coronary microvascular disease) is a condition that affects small blood vessels of the heart, such as, e.g., the arterioles and capillaries. In patients with CMD, the small blood vessels of the heart do not dilate properly in response to stress, leading to inadequate blood flow to the heart, even though the larger coronary arteries may be clear. Symptoms of CMD include angina, reduced exercise tolerance, microvascular ischemia, heart failure, increased risk of major cardiovascular events, and reduced quality of life. However, CMD is often difficult to diagnose as traditional diagnostic tests for CAD (coronary artery disease) may not detect subtle abnormalities in the small blood vessels. Conventionally, the diagnosis of CMD involves a combination of invasive and non-invasive testing, which leads to increased costs, procedure duration, and patient risk.

### BRIEF SUMMARY OF THE INVENTION

In accordance with one or more embodiments, systems and methods for non-invasively determining an assessment of coronary microvascular disease of a patient are provided. Patient data of a patient is received. The patient data comprises patient characteristics and vessel characteristics. An assessment of coronary microvascular disease of the patient is determined based on the patient data using a machine learning based assessment model. Results of the assessment of coronary microvascular disease of the patient are output.

In one embodiment, a value of a microvascular coronary resistance index for the vessel representing a maximal reduction in coronary microvascular resistance of the vessel at maximal stress is determined. The machine learning based assessment model is trained using training patient data of a particular patient annotated with a ground truth value of the microvascular coronary resistance index. The ground truth value of the microvascular coronary resistance index is determined by: receiving a measured FFR (fractional flow reserve) value and a measured CFR (coronary flow reserve) value of a stenosis in a vessel of the particular patient; generating a model of the vessel of the particular patient; determining a ground truth value of a microvascular coronary resistance index using the model of the vessel based on the measured FFR value and the measured CFR value; and outputting the ground truth value of the microvascular coronary resistance index. In one embodiment, the ground truth value of the microvascular coronary resistance index that results in FFR and CFR values that match the measured FFR value and the measured CFR value is determined. In another embodiment, the ground truth value of the microvascular coronary resistance index and a value of a stenosis severity that results in FFR and CFR values that match the measured FFR value and the measured CFR value are determined.

In one embodiment, the vessel characteristics comprise at least one of stress test assessments of the patient, coronary artery disease assessments of the patient, plaque characteristics, or perivascular adipose tissue.

In one embodiment, the patient data comprises PCCT (photon counting computed tomography) images.

These and other advantages of the invention will be apparent to those of ordinary skill in the art by reference to the following detailed description and the accompanying drawings.

### BRIEF DESCRIPTION OF THE DRAWINGS

FIG. 1 shows a method for determining an assessment of coronary microvascular disease of a patient, in accordance with one or more embodiments;

FIG. 2 shows a method for determining ground truth values of a microvascular coronary resistance index, in accordance with one or more embodiments;

FIG. 3 shows a model of a framework for pressure and flow computation, in accordance with one or more embodiments;

FIG. 4 shows an exemplary artificial neural network that may be used to implement one or more embodiments;

FIG. 5 shows a convolutional neural network that may be used to implement one or more embodiments;

FIG. 6 shows a data flow diagram for using a generative adversarial network to implement one or more embodiments;

FIG. 7 shows a schematic structure of a recurrent machine learning model that may be used to implement one or more embodiments; and

FIG. 8 shows a high-level block diagram of a computer that may be used to implement one or more embodiments.

### DETAILED DESCRIPTION

The present invention generally relates to methods and systems for machine learning based non-invasive assessment of coronary microvascular disease from PCCT images. Embodiments of the present invention are described herein to give a visual understanding of such methods and systems. A digital image is often composed of digital representations of one or more objects (or shapes). The digital representation of an object is often described herein in terms of identifying and manipulating the objects. Such manipulations are virtual manipulations accomplished in the memory or other circuitry/hardware of a computer system. Accordingly, is to be understood that embodiments of the present invention may be performed within a computer system using data stored within the computer system. Further, reference herein to pixels of an image may refer equally to voxels of an image and vice versa.

Embodiments described herein provide for a machine learning based assessment model for the non-invasive assessment of coronary microvascular disease. The machine learning based assessment model predicts a microvascular coronary resistance index MCRI_{ML} for each epicardial artery representing the maximal reduction in coronary microvascular resistance of that epicardial artery at maximal stress. Advantageously, embodiments described herein provide for assessment of coronary microvascular disease non-invasively, resulting in reduced costs, procedure duration, and risks as compared with conventional approaches.

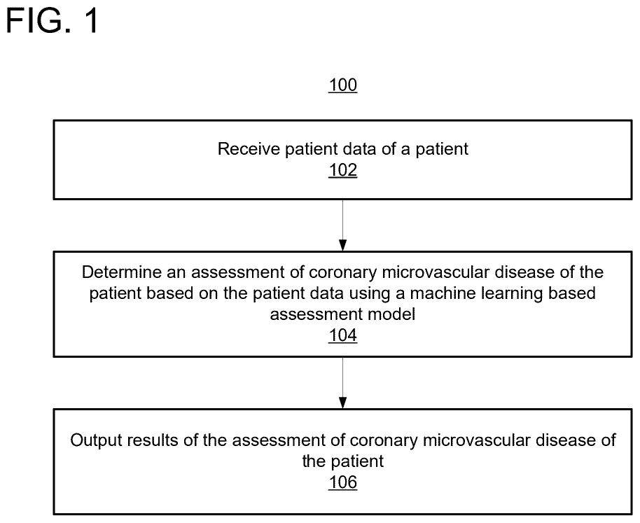

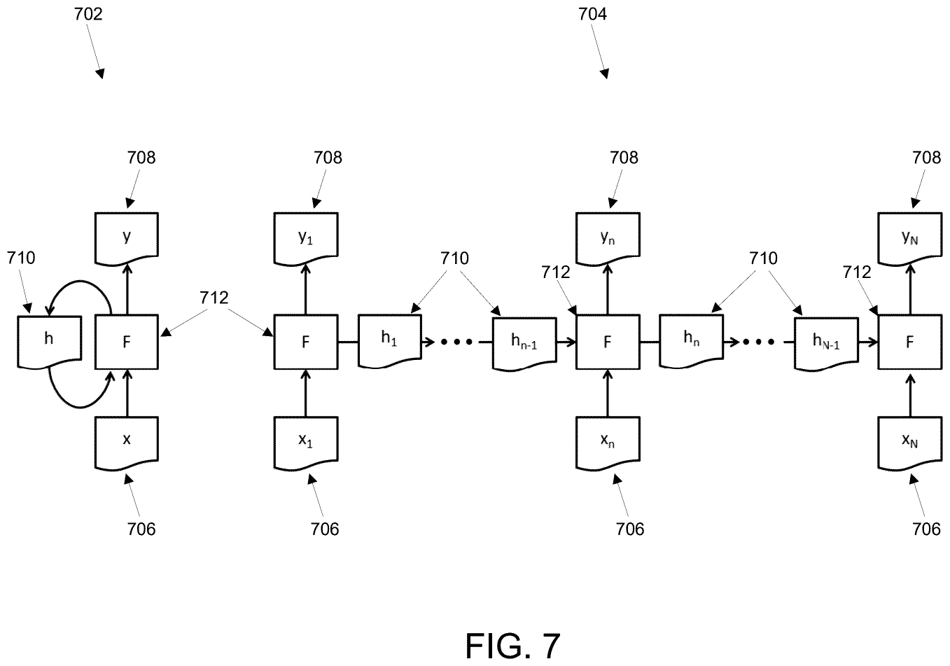

FIG. 1 shows a method 100 for determining an assessment of coronary microvascular disease of a patient, in accordance with one or more embodiments. The steps and sub-steps of method 100 may be performed by one or more suitable computing devices, such as, e.g., computer 702 of FIG. 7.

At step 102 of FIG. 1, patient data of a patient is received. The patient data comprises, for example, patient characteristics and vessel characteristics. In one embodiment, the patient characteristics may comprise, for example, gender, age, body fat, lab tests (e.g., assessing dyslipidemia, inflammation, etc.), other pathologies (e.g., diabetes, hypertension, etc.), patient history, etc. of the patient. In one embodiment, the vessel characteristics may comprise, for example, data relating to, e.g., stress test assessments of the patient at a myocardial segment level (e.g., stress ECG (echocardiography), static/dynamic CTP (computed tomography perfusion), perfusion MRI (magnetic resonance imaging), nuclear imaging), epicardial CAD assessments of the patient (e.g., indicating a severity of stenoses (e.g., percent DS (degenerative spondylolisthesis), stenosis length), plaque characteristics, perivascular adipose tissue, etc. of coronary vessels of the patient. However, the patient characteristics and vessel characteristics may comprise any other suitable patient characteristic and vessel characteristic data of the patient respectively.

The patient data may be represented in any suitable format. For example, the patient data may comprise text-based patient data and/or non-text-based patient data. The text-based patient data may comprise, e.g., clinical reports (e.g., radiology reports), demographic information, vital signs, medical history, family history, laboratory results, measurements and information extracted from medical images, etc. of the patient. The non-text-based patient data may comprise, e.g., one or more medical images depicting one or more anatomical objects of the patient, such as, e.g., vessel and/or any other anatomical object of interest (organs, bones, tumors, etc.). In one embodiment, the one or more medical images may comprise PCCT images of the patient. However, the one or more medical images may be of any suitable modality, such as, e.g., CT (computed tomography), MRI (magnetic resonance imaging), US (ultrasound), x-ray, or any other medical imaging modality or combinations of medical imaging modalities. The one or more medical images may comprise 2D (two dimensional) images and/or 3D (three dimensional) volumes.

The patient data may be received, for example, by directly receiving the patient data from an image acquisition device (e.g., image acquisition device 714 of FIG. 7) as the patient data is acquired, by loading the patient data from a storage or memory of a computer system (e.g., storage 712 or memory 710 of computer 702 of FIG. 7), or by receiving the patient data from a remote computer system (e.g., computer 702 of FIG. 7). Such a computer system or remote computer system may comprise one or more patient databases, such as, e.g., an EHR (electronic health record), EMR (electronic medical record), PHR (personal health record), HIS (health information system), RIS (radiology information system), PACS (picture archiving and communication system), LIMS (laboratory information management system), or any other suitable database or system.

At step 104 of FIG. 1, an assessment of coronary microvascular disease of the patient is determined based on the patient data using a machine learning based assessment model. In one embodiment, the assessment of coronary microvascular disease results in a microvascular coronary resistance index MCRI_{ML} for the vessel. The vessel may be, for example, an epicardial artery or any other vessel having a diameter greater than 400 micrometers. The microvascular coronary resistance index MCRI_{ML} represents the maximal reduction in coronary microvascular resistance of that artery at maximal stress. The results of the assessment of coronary microvascular disease may be represented in any other suitable format.

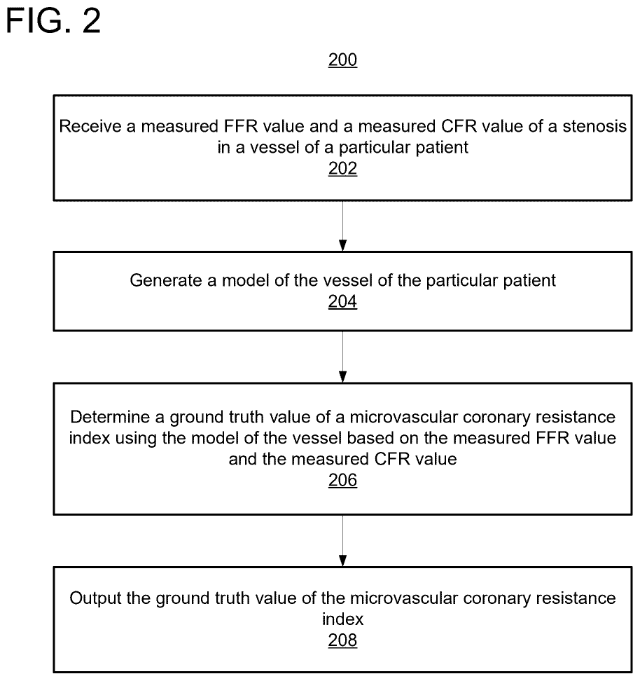

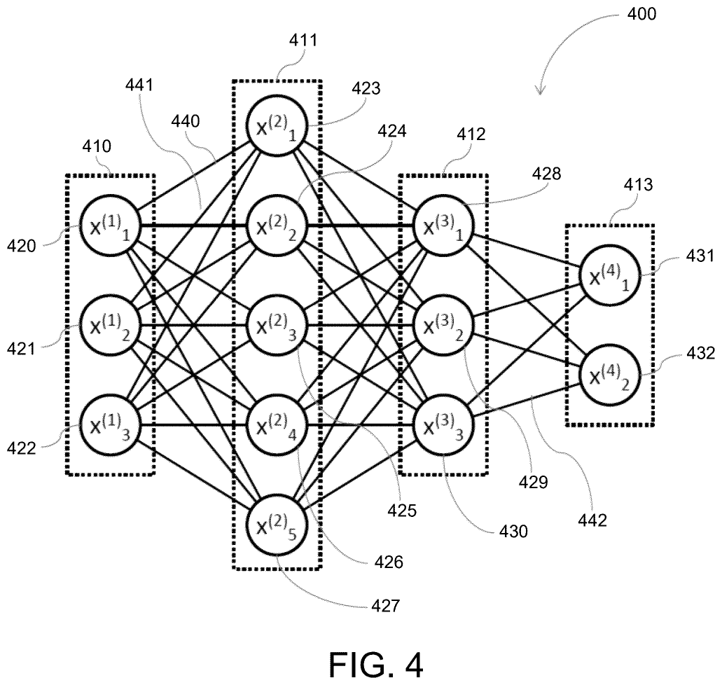

The machine learning based assessment model may be implemented according to any suitable machine learning based architecture, e.g., based on a type of the patient data. The machine learning based assessment model receives as input the patient data and generates as output the results of the assessment of coronary microvascular disease (e.g., the microvascular coronary resistance index MCRI_{ML}). The machine learning based assessment model is trained during an offline or training stage using annotated training patient data. In one embodiment, ground truth microvascular coronary resistance index MCRI_{ML} values are generated for the annotated training patient data according to method 200 of FIG. 2. Once trained, the machine learning based assessment model is applied to determine the assessment of coronary microvascular disease of the patient, e.g., to perform step 104 of FIG. 4.

At step 106 of FIG. 1, results of the assessment of coronary microvascular disease of the patient are output. For example, the results of the assessment of coronary microvascular disease of the patient can be output by displaying the results on a display device of a computer system (e.g., I/O 708 of computer 702 of FIG. 7), storing the results on a memory or storage of a computer system (e.g., memory 710 or storage 712 of computer 702 of FIG. 7), or by transmitting the results to a remote computer system (e.g., computer 702 of FIG. 7).

FIG. 2 shows a method 200 for determining ground truth values of a microvascular coronary resistance index MCRI_{ML}, in accordance with one or more embodiments. The steps and sub-steps of method 100 may be performed by one or more suitable computing devices, such as, e.g., computer 702 of FIG. 7. Method 200 may be repeatedly performed for each particular patient of a patient population for generating training data for training the machine learning based assessment model.

At step 202 of FIG. 2, a measured FFR (fractional flow reserve) value and a measured CFR (coronary flow reserve) value of a stenosis in a vessel of a particular patient are received.

The measured FFR value may be invasively measured using a catheter equipped with a pressure sensor to measure the blood pressure distal to stenosis and the blood pressure proximal to the stenosis while the particular patient is at hyperemia. The measured FFR value is then calculated according to Equation (1):

$$FFR = \frac{{P}_{d}}{{P}_{a}} \qquad ( 1 )$$

where P_{d} is the blood pressure distal to the stenosis and Pₐ is the blood pressure proximal to the stenosis.

The measured CFR value may be invasively measured using a catheter equipped with a vascular doppler to measure the blood flow of the stenosis while the patient is at a hyperemia state and the blood flow of the stenosis while the patient is at a rest state. Alternatively, the measured CFR value may be measured from perfusion imaging (e.g., PET (positron emission tomography), echocardiography, intracoronary doppler ultrasound, MRI, CTP (computed tomography perfusion), etc.) using, e.g., well-known approaches. The measured CFR value is then calculated according to Equation (2):

$$CFR = \frac{{Q}_{rest}}{{Q}_{hyperemia}} \qquad ( 2 )$$

where Qᵣₑₛₜ is the blood flow of the stenosis while the patient is at a rest state and Q_{hyperemia} is the blood flow of the stenosis while the patient is at a hyperemia state.

The measured FFR value and the measured CFR value may be received, for example, by directly receiving the data from an pressure sensor or vascular doppler while the values are acquired and computing the measured FFR value and the measured CFR value, by loading the values from a storage or memory of a computer system (e.g., storage 712 or memory 710 of computer 702 of FIG. 7), or by receiving the values from a remote computer system (e.g., computer 702 of FIG. 7).

At step 204 of FIG. 2, a model of the vessel of the particular patient is generated. The model is a 3D model generated using previously acquired medical images of the vessel of the patient. In one embodiment, the medical images are CTA (computed tomography angiography) medical images of the patient. However, the medical images may be of any other suitable modality or modalities (e.g., coronary angiography images). The model may be generated using any suitable (e.g., well-known) approach.

At step 206 of FIG. 2, a ground truth value of a microvascular coronary resistance index is determined using the model of the vessel based on the measured FFR value and the measured CFR value. The value of a microvascular coronary resistance index MCRI_{ML} is determined using a framework for pressure and flow computation. The framework may be implemented using CFD (computational fluid dynamics) framework and/or a machine-learning based framework according to, e.g., well-known methods. The framework is first run to determine pressure and flow in the coronary arteries when the patient is at rest, by employing autoregulation for calibrating various hemodynamic parameters to match the expected flow through each epicardial artery. The framework is also run to determine pressure and flow in the coronary arteries while the patient is in a hyperemic state. A standard personalization procedure may be employed for all vessels for which FFR and CFR are not known. For vessels for which FFR and CFR are known, model parameters are calibrated to match the measured FFR and CFR values.

Given the model of the vessel, the value of a microvascular coronary resistance index (i.e., the factor by which the microvascular resistance decreases when the patient state changes from a rest state to a maximal hyperemia state) is the only unknown. Hence, for the vessel, two values (i.e., the measured FFR value and the measured CFR value) are known and the value of the microvascular coronary resistance index MCRI_{ML} is the only unknown, leading to an overdetermined system.

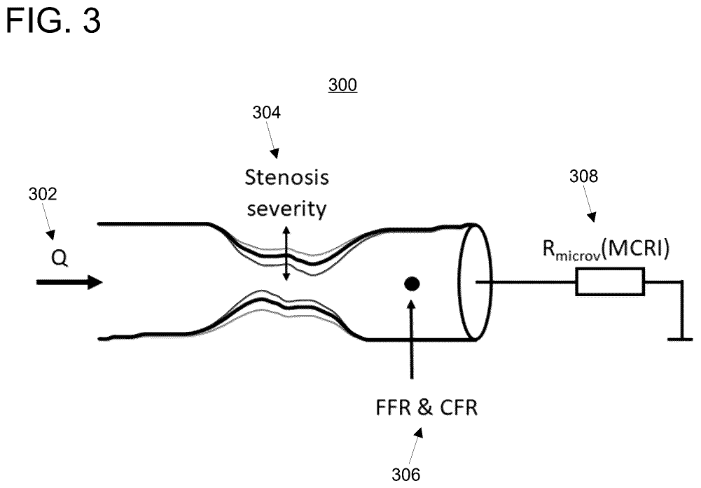

The generating of the model of the vessel may have small errors. Additionally, certain geometric properties (e.g., minimal lumen diameter) have a large influence on hemodynamic indices such as, e.g., FFR and CFR. Accordingly, in one embodiment, a second unknown variable is introduced, the stenosis severity, allowing for small adjustments of the model of the vessel in the region with higher certainty—the stenotic region. Varying the stenotic severity would calibrate the severity of the entire stenosis. Hence, a well determined system is provided where two values (i.e., the measured FFR value and the measured CFR value) are known and two values (i.e., the microvascular coronary resistance index MCRI_{ML} and the stenosis severity) are unknown. The framework for pressure and flow computation with known values of the measured FFR value and the measured CFR and unknown values of the microvascular coronary resistance index MCRI_{ML} and the stenosis severity may be modeled according to FIG. 3.

FIG. 3 shows a model 300 of a framework for pressure and flow computation, in accordance with one or more embodiments. In model 300, the framework for pressure and flow computation (e.g., a CFD and/or machine learning based framework) determines values of microvascular coronary resistance index MCRI_{ML} 308 and stenosis severity 304 as calibrated parameters to match the measured FFR and CFR values 306. Accordingly, the values of microvascular coronary resistance index MCRI_{ML} 308 and stenosis severity 304 that result in the measured FFR and CFR values 306 are determined as the ground truth value of the microvascular coronary resistance index.

Referring back to method 200 of FIG. 2, at step 208, the ground truth value of the microvascular coronary resistance index is output. For example, the ground truth value of the microvascular coronary resistant index can be output by displaying the ground truth value on a display device of a computer system (e.g., I/O 708 of computer 702 of FIG. 7), storing the ground truth value on a memory or storage of a computer system (e.g., memory 710 or storage 712 of computer 702 of FIG. 7), or by transmitting the ground truth value to a remote computer system (e.g., computer 702 of FIG. 7).

In one embodiment, the ground truth value of the microvascular coronary resistance index is used to annotate corresponding patient data of the particular patient to generate annotated training data and the annotated training data is used for training a machine learning assessment model for determining an assessment of contrary microvascular disease of a patient, e.g., to perform step 104 of FIG. 1.

In one embodiment, a generative adversarial model or network may be used to generate synthetic image in one modality from images of another modality (e.g., to generate coronary angiography images from PCCT images). The synthetic images may be used to train different machine learning models for different imaging modalities.

Advantageously, the use of PCCT images in the patient data improves the accuracy for determining an assessment of microvascular disease of the patient in accordance with embodiments described herein. In particular, PCCT images allow for a more precise generation of a model of vessel and allow for visualizing a coronary tree with a larger number of branches, which in turn allows for a more precise mapping of CFR (determined from perfusion imaging) to epicardial arteries.

A further advantage of the embodiments described herein is the determination of a non-invasive, vessel specific, and continuous microvascular coronary resistance index, which alongside epicardial hemodynamic indices (e.g., FFR and CFR) allow for a more informed decision regarding diagnosis and treatment of CAD. The microvascular coronary resistance index could be employed in hemodynamics computations to allow for a more precise non-invasive and concomitant assessment of epicardial and microvascular disease.

Embodiments described herein are described with respect to the claimed systems as well as with respect to the claimed methods. Features, advantages or alternative embodiments herein can be assigned to the other claimed objects and vice versa. In other words, claims and embodiments for the systems can be improved with features described or claimed in the context of the respective methods. In this case, the functional features of the method are implemented by physical units of the system.

Furthermore, certain embodiments described herein are described with respect to methods and systems utilizing trained machine learning models, as well as with respect to methods and systems for providing trained machine learning models. Features, advantages or alternative embodiments herein can be assigned to the other claimed objects and vice versa. In other words, claims and embodiments for providing trained machine learning models can be improved with features described or claimed in the context of utilizing trained machine learning models, and vice versa. In particular, datasets used in the methods and systems for utilizing trained machine learning models can have the same properties and features as the corresponding datasets used in the methods and systems for providing trained machine learning models, and the trained machine learning models provided by the respective methods and systems can be used in the methods and systems for utilizing the trained machine learning models.

In general, a trained machine learning model mimics cognitive functions that humans associate with other human minds. In particular, by training based on training data the machine learning model is able to adapt to new circumstances and to detect and extrapolate patterns. Another term for “trained machine learning model” is “trained function.”

In general, parameters of a machine learning model can be adapted by means of training. In particular, supervised training, semi-supervised training, unsupervised training, reinforcement learning and/or active learning can be used. Furthermore, representation learning (an alternative term is “feature learning”) can be used. In particular, the parameters of the machine learning models can be adapted iteratively by several steps of training. In particular, within the training a certain cost function can be minimized. In particular, within the training of a neural network the backpropagation algorithm can be used.

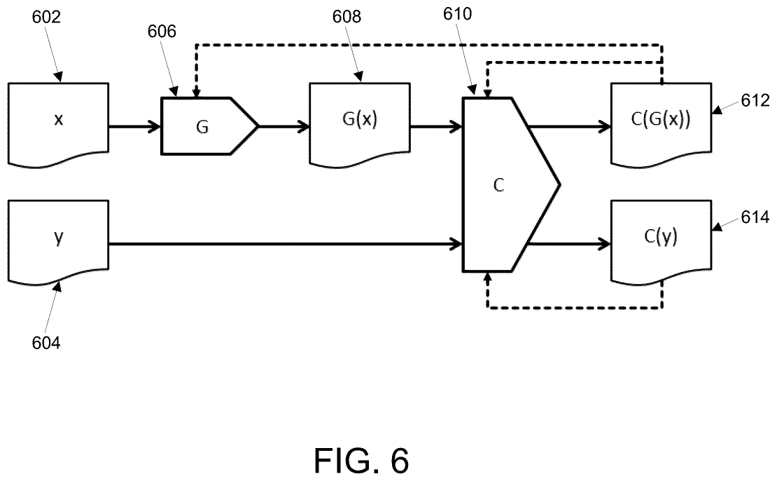

In particular, a machine learning model, such as, e.g., the machine learning based assessment model utilized at step 104 of FIG. 1 and the machine learning based model utilized at step 206 of FIG. 6, can comprise, for example, a neural network, a support vector machine, a decision tree and/or a Bayesian network, and/or the machine learning model can be based on, for example, k-means clustering, Q-learning, genetic algorithms and/or association rules. In particular, a neural network can be, e.g., a deep neural network, a convolutional neural network or a convolutional deep neural network. Furthermore, a neural network can be, e.g., an adversarial network, a deep adversarial network and/or a generative adversarial network.

FIG. 4 shows an embodiment of an artificial neural network 400 that may be used to implement one or more machine learning models described herein. Alternative terms for “artificial neural network” are “neural network”, “artificial neural net” or “neural net”.

The artificial neural network 400 comprises nodes 420, . . . , 432 and edges 440, . . . , 442, wherein each edge 440, . . . , 442 is a directed connection from a first node 420, . . . 432 to a second node 420, . . . , 432. In general, the first node 420, . . . , 432 and the second node 420, . . . , 432 are different nodes 420, . . . , 432, it is also possible that the first node 420, . . . , 432 and the second node 420, . . . , 432 are identical. For example, in FIG. 4 the edge 440 is a directed connection from the node 420 to the node 423, and the edge 442 is a directed connection from the node 430 to the node 432. An edge 440, . . . , 442 from a first node 420, . . . , 432 to a second node 420, . . . , 432 is also denoted as “ingoing edge” for the second node 420, . . . , 432 and as “outgoing edge” for the first node 420, . . . , 432.

In this embodiment, the nodes 420, . . . , 432 of the artificial neural network 400 can be arranged in layers 410, . . . , 413, wherein the layers can comprise an intrinsic order introduced by the edges 440, . . . , 442 between the nodes 420, . . . , 432. In particular, edges 440, . . . , 442 can exist only between neighboring layers of nodes. In the displayed embodiment, there is an input layer 410 comprising only nodes 420, . . . , 422 without an incoming edge, an output layer 413 comprising only nodes 431, 432 without outgoing edges, and hidden layers 411, 412 in-between the input layer 410 and the output layer 413. In general, the number of hidden layers 411, 412 can be chosen arbitrarily. The number of nodes 420, . . . , 422 within the input layer 410 usually relates to the number of input values of the neural network, and the number of nodes 431, 432 within the output layer 413 usually relates to the number of output values of the neural network.

In particular, a (real) number can be assigned as a value to every node 420, . . . , 432 of the neural network 400. Here, x⁽ⁿ⁾ᵢ denotes the value of the i-th node 420, . . . , 432 of the n-th layer 410, . . . , 413. The values of the nodes 420, . . . , 422 of the input layer 410 are equivalent to the input values of the neural network 400, the values of the nodes 431, 432 of the output layer 413 are equivalent to the output value of the neural network 400. Furthermore, each edge 440, . . . , 442 can comprise a weight being a real number, in particular, the weight is a real number within the interval [−1, 1] or within the interval [0, 1]. Here, w^{(m,n)}ᵢⱼ denotes the weight of the edge between the i-th node 420, . . . , 432 of the m-th layer 410, . . . , 413 and the j-th node 420, . . . , 432 of the n-th layer 410, . . . , 413. Furthermore, the abbreviation w⁽ⁿ⁾_{i,j} is defined for the weight w^{(n,n+1)}_{i,j}.

In particular, to calculate the output values of the neural network 400, the input values are propagated through the neural network. In particular, the values of the nodes 420, . . . , 432 of the (n+1)-th layer 410, . . . , 413 can be calculated based on the values of the nodes 420, 432 of the n-th layer 410, . . . , 413 by

$${x}^{{( n + 1 )}_{j}} = f ( {\sum }_{i} {x}^{{( n )}_{i}} \cdot {w}^{{( n )}_{i , j}} ) .$$

Herein, the function f is a transfer function (another term is “activation function”). Known transfer functions are step functions, sigmoid function (e.g., the logistic function, the generalized logistic function, the hyperbolic tangent, the Arctangent function, the error function, the smoothstep function) or rectifier functions. The transfer function is mainly used for normalization purposes.

In particular, the values are propagated layer-wise through the neural network, wherein values of the input layer 410 are given by the input of the neural network 400, wherein values of the first hid-den layer 411 can be calculated based on the values of the input layer 410 of the neural network, wherein values of the second hidden layer 412 can be calculated based in the values of the first hidden layer 411, etc.

In order to set the values w^{(m,n)}_{i,j} for the edges, the neural network 400 has to be trained using training data. In particular, training data comprises training input data and training output data (denoted as tᵢ). For a training step, the neural network 400 is applied to the training input data to generate calculated output data. In particular, the training data and the calculated output data comprise a number of values, said number being equal with the number of nodes of the output layer.

In particular, a comparison between the calculated output data and the training data is used to recursively adapt the weights within the neural network 400 (backpropagation algorithm). In particular, the weights are changed according to

$${w}^{{′ ( n )}_{i , j}} = {w}^{{( n )}_{i , j}} - y \cdot {δ}^{{( n )}_{j}} \cdot {x}^{{( n )}_{i}}$$

wherein γ is a learning rate, and the numbers δ⁽ⁿ⁾ⱼ can be recursively calculated as

$${δ}^{{( n )}_{j}} = ( {\sum }_{k} {δ}^{{( n + 1 )}_{k}} \cdot {w}^{{( n + 1 )}_{j , k}} ) \cdot {f}^{′} ( {\sum }_{i} {x}^{{( n )}_{i}} \cdot {w}^{{( n )}_{i , j}} )$$

based on δ⁽ⁿ⁺¹⁾ⱼ, if the (n+1)-th layer is not the output layer, and

$${δ}^{{( n )}_{j}} = ( {x}^{{( n + 1 )}_{j}} - {t}^{{( n + 1 )}_{j}} ) \cdot {f}^{′} ( {x}^{{( n )}_{i}} \cdot {w}^{{( n )}_{i , j}} )$$

if the (n+1)-th layer is the output layer 413, wherein f′ is the first derivative of the activation function, and t⁽ⁿ⁺¹⁾ⱼ is the comparison training value for the j-th node of the output layer 413.

A convolutional neural network is a neural network that uses a convolution operation instead general matrix multiplication in at least one of its layers (so-called “convolutional layer”). In particular, a convolutional layer performs a dot product of one or more convolution kernels with the convolutional layer's input data/image, wherein the entries of the one or more convolution kernel are the parameters or weights that are adapted by training. In particular, one can use the Frobenius inner product and the ReLU activation function. A convolutional neural network can comprise additional layers, e.g., pooling layers, fully connected layers, and normalization layers.

By using convolutional neural networks input images can be processed in a very efficient way, because a convolution operation based on different kernels can extract various image features, so that by adapting the weights of the convolution kernel the relevant image features can be found during training. Furthermore, based on the weight-sharing in the convolutional kernels less parameters need to be trained, which prevents overfitting in the training phase and allows to have faster training or more layers in the network, improving the performance of the network.

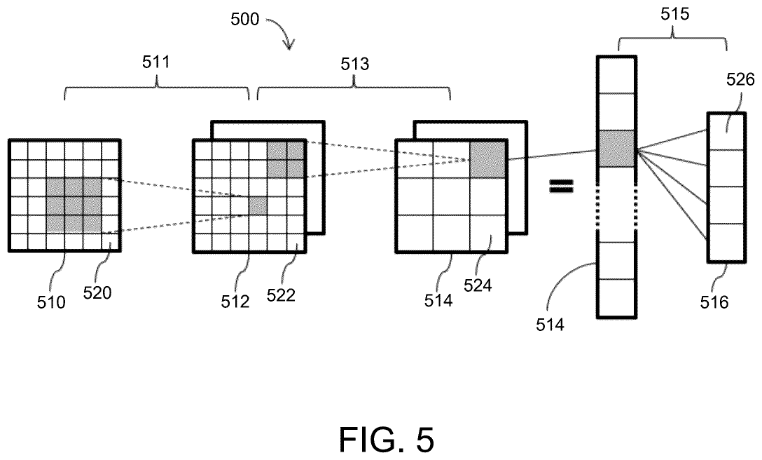

FIG. 5 shows an embodiment of a convolutional neural network 500 that may be used to implement one or more machine learning models described herein. In the displayed embodiment, the convolutional neural network comprises 500 an input node layer 510, a convolutional layer 511, a pooling layer 513, a fully connected layer 514 and an output node layer 516, as well as hidden node layers 512, 514. Alternatively, the convolutional neural network 500 can comprise several convolutional layers 511, several pooling layers 513 and several fully connected layers 515, as well as other types of layers. The order of the layers can be chosen arbitrarily, usually fully connected layers 515 are used as the last layers before the output layer 516.

In particular, within a convolutional neural network 500 nodes 520, 522, 524 of a node layer 510, 512, 514 can be considered to be arranged as a d-dimensional matrix or as a d-dimensional image. In particular, in the two-dimensional case the value of the node 520, 522, 524 indexed with i and j in the n-th node layer 510, 512, 514 can be denoted as x(n)[i, j]. However, the arrangement of the nodes 520, 522, 524 of one node layer 510, 512, 514 does not have an effect on the calculations executed within the convolutional neural network 500 as such, since these are given solely by the structure and the weights of the edges.

A convolutional layer 511 is a connection layer between an anterior node layer 510 (with node values x(n−1)) and a posterior node layer 512 (with node values x(n)). In particular, a convolutional layer 511 is characterized by the structure and the weights of the incoming edges forming a convolution operation based on a certain number of kernels. In particular, the structure and the weights of the edges of the convolutional layer 511 are chosen such that the values x(n) of the nodes 522 of the posterior node layer 512 are calculated as a convolution x(n)=K*x(n−1) based on the values x(n−1) of the nodes 520 anterior node layer 510, where the convolution * is defined in the two-dimensional case as

$${x}_{k}^{( n )} [ i , j ] = ( K * {x}^{( n - 1 )} ) [ i , j ] = {\sum }_{{i}^{′}} \cdot {\sum }_{{j}^{′}} K [ {i}^{′} , {j}^{′} ] \cdot {x}^{( n - 1 )} [ i - {i}^{′} , j - {j}^{′} ] .$$

Here the kernel K is a d-dimensional matrix (in this embodiment, a two-dimensional matrix), which is usually small compared to the number of nodes 520, 522 (e.g., a 3×3 matrix, or a 5×5 matrix). In particular, this implies that the weights of the edges in the convolution layer 511 are not independent, but chosen such that they produce said convolution equation. In particular, for a kernel being a 3×3 matrix, there are only 9 independent weights (each entry of the kernel matrix corresponding to one independent weight), irrespectively of the number of nodes 520, 522 in the anterior node layer 510 and the posterior node layer 512.

In general, convolutional neural networks 500 use node layers 510, 512, 514 with a plurality of channels, in particular, due to the use of a plurality of kernels in convolutional layers 511. In those cases, the node layers can be considered as (d+1)-dimensional matrices (the first dimension indexing the channels). The action of a convolutional layer 511 is then a two-dimensional example defined as

$${x}^{{( n )}_{b}} [ i , j ] = {\sum }_{a} {K}_{a , b} * {x}^{{( n - 1 )}_{a}} [ i , j ] = {\sum }_{a} {\sum }_{{i}^{′}} {\sum }_{{j}^{′}} {K}_{a , b} [ {i}^{′} , {j}^{′} ] \cdot {x}^{{( n - 1 )}_{a}} [ i - {i}^{′} , j - {j}^{′} ]$$

where x⁽ⁿ⁻¹⁾ₐ corresponds to the a-th channel of the anterior node layer 510, x⁽ⁿ⁾_{b} corresponds to the b-th channel of the posterior node layer 512 and K_{a,b} corresponds to one of the kernels. If a convolutional layer 511 acts on an anterior node layer 510 with A channels and outputs a posterior node layer 512 with B channels, there are A·B independent d-dimensional kernels K_{a,b}.

In general, in convolutional neural networks 500 activation functions are used. In this embodiment re ReLU (acronym for “Rectified Linear Units”) is used, with R(z)=max(0, z), so that the action of the convolutional layer 511 in the two-dimensional example is

$${x}^{{( n )}_{b}} [ i , j ] = R ( {\sum }_{a} ( {K}_{a , b} * {x}^{{( n - 1 )}_{a}} ) [ i , j ] ) = R ( {\sum }_{a} {\sum }_{{i}^{′}} {\sum }_{{j}^{′}} {K}_{a , b} [ {i}^{′} , {j}^{′} ] \cdot {x}^{{( n - 1 )}_{a}} [ i - {i}^{′} , j - {j}^{′} ] )$$

It is also possible to use other activation functions, e.g., ELU (acronym for “Exponential Linear Unit”), LeakyReLU, Sigmoid, Tanh or Softmax.

In the displayed embodiment, the input layer 510 comprises 36 nodes 520, arranged as a two-dimensional 6×6 matrix. The first hidden node layer 512 comprises 72 nodes 522, arranged as two two-dimensional 6×6 matrices, each of the two matrices being the result of a convolution of the values of the input layer with a 3×3 kernel within the convolutional layer 511. Equivalently, the nodes 522 of the first hidden node layer 512 can be interpreted as arranged as a three-dimensional 2×6×6 matrix, wherein the first dimension correspond to the channel dimension.

The advantage of using convolutional layers 511 is that spatially local correlation of the input data can exploited by enforcing a local connectivity pattern between nodes of adjacent layers, in particular by each node being connected to only a small region of the nodes of the preceding layer.

A pooling layer 513 is a connection layer between an anterior node layer 512 (with node values x(n−1)) and a posterior node layer 514 (with node values x(n)). In particular, a pooling layer 513 can be characterized by the structure and the weights of the edges and the activation function forming a pooling operation based on a non-linear pooling function f. For example, in the two-dimensional case the values x(n) of the nodes 524 of the posterior node layer 514 can be calculated based on the values x(n−1) of the nodes 522 of the anterior node layer 512 as

x⁽ⁿ⁾^{_{b}}[i,j]=f(x⁽ⁿ⁻¹⁾[id₁,jd₂], . . . ,x⁽ⁿ⁻¹⁾^{_{b}}[(i+1)d₁−1,(j+1)d₂−1])

In other words, by using a pooling layer 513 the number of nodes 522, 524 can be reduced, by re-placing a number d1·d2 of neighboring nodes 522 in the anterior node layer 512 with a single node 522 in the posterior node layer 514 being calculated as a function of the values of said number of neighboring nodes. In particular, the pooling function f can be the max-function, the average or the L2-Norm. In particular, for a pooling layer 513 the weights of the incoming edges are fixed and are not modified by training.

The advantage of using a pooling layer 513 is that the number of nodes 522, 524 and the number of parameters is reduced. This leads to the amount of computation in the network being reduced and to a control of overfitting.

In the displayed embodiment, the pooling layer 513 is a max-pooling layer, replacing four neighboring nodes with only one node, the value being the maximum of the values of the four neighboring nodes. The max-pooling is applied to each d-dimensional matrix of the previous layer; in this embodiment, the max-pooling is applied to each of the two two-dimensional matrices, reducing the number of nodes from 72 to 18.

In general, the last layers of a convolutional neural network 500 are fully connected layers 515. A fully connected layer 515 is a connection layer between an anterior node layer 514 and a posterior node layer 516. A fully connected layer 513 can be characterized by the fact that a majority, in particular, all edges between nodes 514 of the anterior node layer 514 and the nodes 516 of the posterior node layer are present, and wherein the weight of each of these edges can be adjusted individually.

In this embodiment, the nodes 524 of the anterior node layer 514 of the fully connected layer 515 are displayed both as two-dimensional matrices, and additionally as non-related nodes (indicated as a line of nodes, wherein the number of nodes was reduced for a better presentability). This operation is also denoted as “flattening”. In this embodiment, the number of nodes 526 in the posterior node layer 516 of the fully connected layer 515 smaller than the number of nodes 524 in the anterior node layer 514. Alternatively, the number of nodes 526 can be equal or larger.

Furthermore, in this embodiment the Softmax activation function is used within the fully connected layer 515. By applying the Softmax function, the sum the values of all nodes 526 of the output layer 516 is 1, and all values of all nodes 526 of the output layer 516 are real numbers between 0 and 1. In particular, if using the convolutional neural network 500 for categorizing input data, the values of the output layer 516 can be interpreted as the probability of the input data falling into one of the different categories.

In particular, convolutional neural networks 500 can be trained based on the backpropagation algorithm. For preventing overfitting, methods of regularization can be used, e.g., dropout of nodes 520, . . . , 524, stochastic pooling, use of artificial data, weight decay based on the L1 or the L2 norm, or max norm constraints.

According to an aspect, the machine learning model may comprise one or more residual networks (ResNet). In particular, a ResNet is an artificial neural network comprising at least one jump or skip connection used to jump over at least one layer of the artificial neural network. In particular, a ResNet may be a convolutional neural network comprising one or more skip connections respectively skipping one or more convolutional layers. According to some examples, the ResNets may be represented as m-layer ResNets, where m is the number of layers in the corresponding architecture and, according to some examples, may take values of 34, 50, 101, or 152. According to some examples, such an m-layer ResNet may respectively comprise (m−2)/2 skip connections.

A skip connection may be seen as a bypass which directly feeds the output of one preceding layer over one or more bypassed layers to a layer succeeding the one or more bypassed layers. Instead of having to directly fit a desired mapping, the bypassed layers would then have to fit a residual mapping “balancing” the directly fed output.

Fitting the residual mapping is computationally easier to optimize than the directed mapping. What is more, this alleviates the problem of vanishing/exploding gradients during optimization upon training the machine learning models: if a bypassed layer runs into such problems, its contribution may be skipped by regularization of the directly fed output. Using ResNets thus brings about the advantage that much deeper networks may be trained.

A generative adversarial model (an acronym is GA model) comprises a generative function and a discriminative function, wherein the generative function creates synthetic data, and the discriminative function distinguishes between synthetic and real data. By training the generative function and/or the discriminative function on the one hand the generative function is configured to create synthetic data which is incorrectly classified by the discriminative function as real, on the other hand the discriminative function is configured to distinguish between real data and synthetic data generated by the generative function. In the notion of game theory, a generative adversarial model can be interpreted as a zero-sum game. The training of the generative function and/or of the discriminative function is based, in particular, on the minimization of a cost function.

By using a GA model, based on a set of training data synthetic data can be generated that has the same characteristics as the training data set. The training of the GA model can be based on data not being annotated (unsupervised learning), so that there is low effort in training a GA model.

FIG. 6 shows a data flow diagram according to an embodiment for using a generative adversarial network for creating synthetic output data G(x) 608 based on input data x 602 that is indistinguishable from real output data y 604, in accordance with one or more embodiments. The synthetic output data G(x) 608 has the same structure as the real output data y 604, but its content is not derived from real world data.

The generative adversarial network comprises a generator function G 606 and a classifier function C 610 which are trained jointly. The task of the generator function G 606 is to provide realistic synthetic output data G(x) 608 based on input data x 602, and the task of the classifier function C 610 is to distinguish between real output data y 604 and synthetic output data G(x) 608. In particular, the output of the classifier function C 610 is a real number between 0 and 1 corresponding to the probability of the input value being real data, so that an ideal classifier function would calculate an output value of C(y) 614≈1 for real data y 604 and C(G(x)) 612≈0 for synthetic data G(x) 608.

Within the training process, parameters of the generator function G 606 are adapted so that the synthetic output data G(x) 608 has the same characteristics as real output data y 604, so that the classifier function C 610 cannot distinguish between real and synthetic data anymore. At the same time, parameters of the classifier function C 610 are adapted so that it distinguishes between real and synthetic data in the best possible way. Here, the training relies on pairs comprising input data x 602 and the corresponding real output data y 604. Within a single training step, the generator function G 606 is applied to the input data x 602 for generating synthetic output data G(x) 608. Furthermore, the classifier function C 610 is applied to the real output data y 604 for generating a first classification result C(y) 614. Additionally, the classifier function C 610 is applied to the synthetic output data G(x) 608 for generating a second classification result C(G(x)) 612.

Adapting the parameters of the generative function G 606 and the classifier function C 610 is based on minimizing a cost function by using the backpropagation algorithm, respectively. In this embodiment, the cost function K_{C} for the classifier function C 610 is K_{C}∝−BCE(C(y), 1)−BCE(C(G(x), 0), wherein BCE denotes the binary cross entropy defined as BCE(z, z′)=z′·log (z)+(1−z′)·log (1−z). By using this cost function, both wrongly classifying real output data as synthetic (indicated by C(y)≈0) and wrongly classifying synthetic output data as real (indicated as C(G(x)) 612≈1) increases the cost function K_{C} to be minimized. Furthermore, the cost function K_{G} for the generator function G 606 is K_{G}∝−BCE(C(G(x), 1)=−log (C(G(x). By using this cost function, correctly classified synthetic output data (indicated as C(G(x)) 612≈0) leads to an increase of the cost function K_{G} to be minimized.

In particular, a recurrent machine learning model is a machine learning model whose output does not only depend on the input value and the parameters of the machine learning model adapted by the training process, but also on a hidden state vector, wherein the hidden state vector is based on previous inputs used on for the recurrent machine learning model. In particular, the recurrent machine learning model can comprise additional storage states or additional structures that incorporate time delays or comprise feedback loops.

In particular, the underlying structure of a recurrent machine learning model can be a neural network, which can be denoted as recurrent neural network. Such a recurrent neural network can be described as an artificial neural network where connections between nodes form a directed graph along a temporal sequence. In particular, a recurrent neural network can be interpreted as directed acyclic graph. In particular, the recurrent neural network can be a finite impulse recurrent neural network or an infinite impulse recurrent neural network (wherein a finite impulse network can be unrolled and replaced with a strictly feedforward neural network, and an infinite impulse network cannot be unrolled and replaced with a strictly feedforward neural network).

In particular, training a recurrent neural network can be based on the BPTT algorithm (acronym for “backpropagation through time”), on the RTRL algorithm (acronym for “real-time recurrent learning”) and/or on genetic algorithms.

By using a recurrent machine learning model input data comprising sequences of variable length can be used. In particular, this implies that the method cannot be used only for a fixed number of input datasets (and needs to be trained differently for every other number of input datasets used as input), but can be used for an arbitrary number of input datasets. This implies that the whole set of training data, independent of the number of input datasets contained in different sequences, can be used within the training, and that training data is not reduced to training data corresponding to a certain number of successive input datasets.

FIG. 7 shows the schematic structure of a recurrent machine learning model F, both in a recurrent representation 702 and in an unfolded representation 704, that may be used to implement one or more machine learning models described herein. The recurrent machine learning model takes as input several input datasets x, x₁, . . . , x_{N} 706 and creates a corresponding set of output datasets y, y₁, . . . , y_{N} 708. Furthermore, the output depends on a so-called hidden vector h, h₁, . . . , h_{N} 710, which implicitly comprises information about input datasets previously used as input for the recurrent machine learning model F 712. By using these hidden vectors h, h₁, . . . , h_{N} 710, a sequentiality of the input datasets can be leveraged.

In a single step of the processing, the recurrent machine learning model F 712 takes as input the hidden vector hₙ₋₁ created within the previous step and an input dataset xₙ. Within this step, the recurrent machine learning model F generates as output an updated hidden vector hₙ and an output dataset yₙ. In other words, one step of processing calculates (yₙ, hₙ)=F(xₙ, hₙ₋₁), or by splitting the recurrent machine learning model F 712 into a part F(y) calculating the output data and F (h) calculating the hidden vector, one step of processing calculates yₙ=F^{(y)}(xₙ, hₙ₋₁) and hₙ=F^{(h)} (xₙ, hₙ₋₁). For the first processing step, h₀ can be chosen randomly or filled with all entries being zero. The parameters of the recurrent machine learning model F 712 that were trained based on training datasets before do not change between the different processing steps.

In particular, the output data and the hidden vector of a processing step depend on all the previous input datasets used in the previous steps. yₙ=F^{(y)} (xₙ, F^{(h)}(xₙ₋₁, hₙ₋₂)) and hₙ=F(h)(xₙ, F^{(h)}(xₙ₋₁, hₙ₋₂)).

Systems, apparatuses, and methods described herein may be implemented using digital circuitry, or using one or more computers using well-known computer processors, memory units, storage devices, computer software, and other components. Typically, a computer includes a processor for executing instructions and one or more memories for storing instructions and data. A computer may also include, or be coupled to, one or more mass storage devices, such as one or more magnetic disks, internal hard disks and removable disks, magneto-optical disks, optical disks, etc.

Systems, apparatuses, and methods described herein may be implemented using computers operating in a client-server relationship. Typically, in such a system, the client computers are located remotely from the server computer and interact via a network. The client-server relationship may be defined and controlled by computer programs running on the respective client and server computers.

Systems, apparatuses, and methods described herein may be implemented within a network-based cloud computing system. In such a network-based cloud computing system, a server or another processor that is connected to a network communicates with one or more client computers via a network. A client computer may communicate with the server via a network browser application residing and operating on the client computer, for example. A client computer may store data on the server and access the data via the network. A client computer may transmit requests for data, or requests for online services, to the server via the network. The server may perform requested services and provide data to the client computer(s). The server may also transmit data adapted to cause a client computer to perform a specified function, e.g., to perform a calculation, to display specified data on a screen, etc. For example, the server may transmit a request adapted to cause a client computer to perform one or more of the steps or functions of the methods and workflows described herein, including one or more of the steps or functions of FIGS. 1-3. Certain steps or functions of the methods and workflows described herein, including one or more of the steps or functions of FIGS. 1-3, may be performed by a server or by another processor in a network-based cloud-computing system. Certain steps or functions of the methods and workflows described herein, including one or more of the steps of FIGS. 1-3, may be performed by a client computer in a network-based cloud computing system. The steps or functions of the methods and workflows described herein, including one or more of the steps of FIGS. 1-3, may be performed by a server and/or by a client computer in a network-based cloud computing system, in any combination.

Systems, apparatuses, and methods described herein may be implemented using a computer program product tangibly embodied in an information carrier, e.g., in a non-transitory machine-readable storage device, for execution by a programmable processor; and the method and workflow steps described herein, including one or more of the steps or functions of FIGS. 1-3, may be implemented using one or more computer programs that are executable by such a processor. A computer program is a set of computer program instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

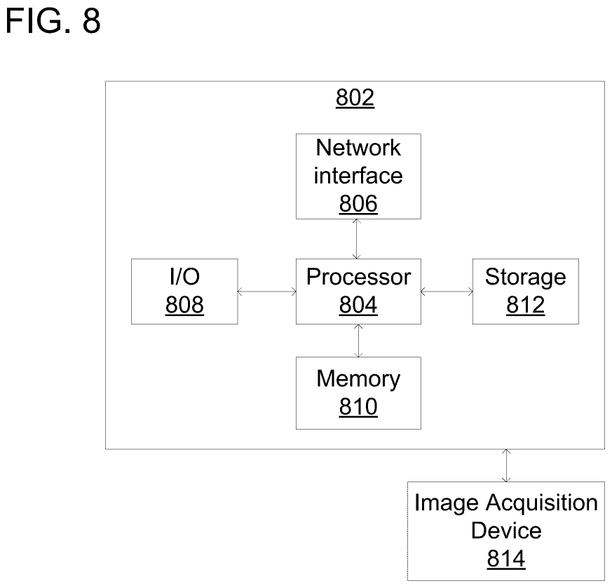

A high-level block diagram of an example computer 802 that may be used to implement systems, apparatuses, and methods described herein is depicted in FIG. 8. Computer 802 includes a processor 804 operatively coupled to a data storage device 812 and a memory 810. Processor 804 controls the overall operation of computer 802 by executing computer program instructions that define such operations. The computer program instructions may be stored in data storage device 812, or other computer readable medium, and loaded into memory 810 when execution of the computer program instructions is desired. Thus, the method and workflow steps or functions of FIGS. 1-3 can be defined by the computer program instructions stored in memory 810 and/or data storage device 812 and controlled by processor 804 executing the computer program instructions. For example, the computer program instructions can be implemented as computer executable code programmed by one skilled in the art to perform the method and workflow steps or functions of FIGS. 1-3. Accordingly, by executing the computer program instructions, the processor 804 executes the method and workflow steps or functions of FIGS. 1-3. Computer 802 may also include one or more network interfaces 806 for communicating with other devices via a network. Computer 802 may also include one or more input/output devices 808 that enable user interaction with computer 802 (e.g., display, keyboard, mouse, speakers, buttons, etc.).

Processor 804 may include both general and special purpose microprocessors, and may be the sole processor or one of multiple processors of computer 802. Processor 804 may include one or more central processing units (CPUs), for example. Processor 804, data storage device 812, and/or memory 810 may include, be supplemented by, or incorporated in, one or more application-specific integrated circuits (ASICs) and/or one or more field programmable gate arrays (FPGAs).

Data storage device 812 and memory 810 each include a tangible non-transitory computer readable storage medium. Data storage device 812, and memory 810, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Input/output devices 808 may include peripherals, such as a printer, scanner, display screen, etc. For example, input/output devices 808 may include a display device such as a cathode ray tube (CRT) or liquid crystal display (LCD) monitor for displaying information to the user, a keyboard, and a pointing device such as a mouse or a trackball by which the user can provide input to computer 802.

An image acquisition device 814 can be connected to the computer 802 to input image data (e.g., medical images) to the computer 802. It is possible to implement the image acquisition device 814 and the computer 802 as one device. It is also possible that the image acquisition device 814 and the computer 802 communicate wirelessly through a network. In a possible embodiment, the computer 802 can be located remotely with respect to the image acquisition device 814.

Any or all of the systems, apparatuses, and methods discussed herein may be implemented using one or more computers such as computer 802.

One skilled in the art will recognize that an implementation of an actual computer or computer system may have other structures and may contain other components as well, and that FIG. 8 is a high level representation of some of the components of such a computer for illustrative purposes.

Independent of the grammatical term usage, individuals with male, female or other gender identities are included within the term.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

The following is a list of non-limiting illustrative embodiments disclosed herein:

Illustrative embodiment 1. A computer-implemented method comprising: receiving patient data of a patient, the patient data comprising patient characteristics and vessel characteristics; determining an assessment of coronary microvascular disease of the patient based on the patient data using a machine learning based assessment model; and outputting results of the assessment of coronary microvascular disease of the patient.

Illustrative embodiment 2. The computer-implemented method of illustrative embodiment 1, wherein determining an assessment of coronary microvascular disease of the patient based on the patient data using a machine learning based assessment model comprises: determining a value of a microvascular coronary resistance index for the vessel representing a maximal reduction in coronary microvascular resistance of the vessel at maximal stress.

Illustrative embodiment 3. The computer-implemented method of illustrative embodiment 2, wherein the machine learning based assessment model is trained using training patient data of a particular patient annotated with a ground truth value of the microvascular coronary resistance index.

Illustrative embodiment 4. The computer-implemented method of illustrative embodiment 3, wherein the ground truth value of the microvascular coronary resistance index is determined by: receiving a measured FFR (fractional flow reserve) value and a measured CFR (coronary flow reserve) value of a stenosis in a vessel of the particular patient; generating a model of the vessel of the particular patient; determining a ground truth value of a microvascular coronary resistance index using the model of the vessel based on the measured FFR value and the measured CFR value; and outputting the ground truth value of the microvascular coronary resistance index.

Illustrative embodiment 5. The computer-implemented method of illustrative embodiment 4, wherein determining a ground truth value of a microvascular coronary resistance index using the model of the vessel based on the measured FFR value and the measured CFR value comprises: determining the ground truth value of the microvascular coronary resistance index that results in FFR and CFR values that match the measured FFR value and the measured CFR value.

Illustrative embodiment 6. The computer-implemented method of any one of illustrative embodiments 4-5, wherein determining a ground truth value of a microvascular coronary resistance index using the model of the vessel based on the measured FFR value and the measured CFR value comprises: determining the ground truth value of the microvascular coronary resistance index and a value of a stenosis severity that results in FFR and CFR values that match the measured FFR value and the measured CFR value.

Illustrative embodiment 7. The computer-implemented method of any one of illustrative embodiments 1-6, wherein the vessel characteristics comprise at least one of stress test assessments of the patient, coronary artery disease assessments of the patient, plaque characteristics, or perivascular adipose tissue.

Illustrative embodiment 8. The computer-implemented method of any one of illustrative embodiments 1-7, wherein the patient data comprises PCCT (photon counting computed tomography) images.

Illustrative embodiment 9. An apparatus comprising: means for receiving patient data of a patient, the patient data comprising patient characteristics and vessel characteristics; means for determining an assessment of coronary microvascular disease of the patient based on the patient data using a machine learning based assessment model; and means for outputting results of the assessment of coronary microvascular disease of the patient.

Illustrative embodiment 10. The apparatus of illustrative embodiment 9, wherein the means for determining an assessment of coronary microvascular disease of the patient based on the patient data using a machine learning based assessment model comprises: means for determining a value of a microvascular coronary resistance index for the vessel representing a maximal reduction in coronary microvascular resistance of the vessel at maximal stress.

Illustrative embodiment 11. The apparatus of illustrative embodiment 10, wherein the machine learning based assessment model is trained using training patient data of a particular patient annotated with a ground truth value of the microvascular coronary resistance index.

Illustrative embodiment 12. The apparatus of illustrative embodiment 11, wherein the ground truth value of the microvascular coronary resistance index is determined by: receiving a measured FFR (fractional flow reserve) value and a measured CFR (coronary flow reserve) value of a stenosis in a vessel of the particular patient; generating a model of the vessel of the particular patient; determining a ground truth value of a microvascular coronary resistance index using the model of the vessel based on the measured FFR value and the measured CFR value; and outputting the ground truth value of the microvascular coronary resistance index.

Illustrative embodiment 13. The apparatus of illustrative embodiment 12, wherein the means for determining a ground truth value of a microvascular coronary resistance index using the model of the vessel based on the measured FFR value and the measured CFR value comprises: means for determining the ground truth value of the microvascular coronary resistance index that results in FFR and CFR values that match the measured FFR value and the measured CFR value.

Illustrative embodiment 14. The apparatus of any one of illustrative embodiments 12-13, wherein the means for determining a ground truth value of a microvascular coronary resistance index using the model of the vessel based on the measured FFR value and the measured CFR value comprises: means for determining the ground truth value of the microvascular coronary resistance index and a value of a stenosis severity that results in FFR and CFR values that match the measured FFR value and the measured CFR value.

Illustrative embodiment 15. A non-transitory computer-readable storage medium comprising instructions which, when executed by a computer, cause the computer to carry out operations comprising: receiving patient data of a patient, the patient data comprising patient characteristics and vessel characteristics; determining an assessment of coronary microvascular disease of the patient based on the patient data using a machine learning based assessment model; and outputting results of the assessment of coronary microvascular disease of the patient.

Illustrative embodiment 16. The non-transitory computer-readable storage medium of illustrative embodiment 15, wherein determining an assessment of coronary microvascular disease of the patient based on the patient data using a machine learning based assessment model comprises: determining a value of a microvascular coronary resistance index for the vessel representing a maximal reduction in coronary microvascular resistance of the vessel at maximal stress.

Illustrative embodiment 17. The non-transitory computer-readable storage medium of illustrative embodiment 16, wherein the machine learning based assessment model is trained using training patient data of a particular patient annotated with a ground truth value of the microvascular coronary resistance index.

Illustrative embodiment 18. The non-transitory computer-readable storage medium of illustrative embodiment 17, wherein the ground truth value of the microvascular coronary resistance index is determined by: receiving a measured FFR (fractional flow reserve) value and a measured CFR (coronary flow reserve) value of a stenosis in a vessel of the particular patient; generating a model of the vessel of the particular patient; determining a ground truth value of a microvascular coronary resistance index using the model of the vessel based on the measured FFR value and the measured CFR value; and outputting the ground truth value of the microvascular coronary resistance index.

Illustrative embodiment 19. The non-transitory computer-readable storage medium of any one of illustrative embodiments 15-18, wherein the vessel characteristics comprise at least one of stress test assessments of the patient, coronary artery disease assessments of the patient, plaque characteristics, or perivascular adipose tissue.

Illustrative embodiment 20. The non-transitory computer-readable storage medium of any one of illustrative embodiments 15-19, wherein the patient data comprises PCCT (photon counting computed tomography) images.

1. A computer-implemented method comprising:
receiving patient data of a patient, the patient data comprising patient characteristics and vessel characteristics;
determining an assessment of coronary microvascular disease of the patient based on the patient data using a machine learning based assessment model; and
outputting results of the assessment of coronary microvascular disease of the patient.

2. The computer-implemented method of claim 1, wherein determining an assessment of coronary microvascular disease of the patient based on the patient data using a machine learning based assessment model comprises:
determining a value of a microvascular coronary resistance index for the vessel representing a maximal reduction in coronary microvascular resistance of the vessel at maximal stress.

3. The computer-implemented method of claim 2, wherein the machine learning based assessment model is trained using training patient data of a particular patient annotated with a ground truth value of the microvascular coronary resistance index.

4. The computer-implemented method of claim 3, wherein the ground truth value of the microvascular coronary resistance index is determined by:
receiving a measured FFR (fractional flow reserve) value and a measured CFR (coronary flow reserve) value of a stenosis in a vessel of the particular patient;
generating a model of the vessel of the particular patient;
determining a ground truth value of a microvascular coronary resistance index using the model of the vessel based on the measured FFR value and the measured CFR value; and
outputting the ground truth value of the microvascular coronary resistance index.

5. The computer-implemented method of claim 4, wherein determining a ground truth value of a microvascular coronary resistance index using the model of the vessel based on the measured FFR value and the measured CFR value comprises:
determining the ground truth value of the microvascular coronary resistance index that results in FFR and CFR values that match the measured FFR value and the measured CFR value.

6. The computer-implemented method of claim 4, wherein determining a ground truth value of a microvascular coronary resistance index using the model of the vessel based on the measured FFR value and the measured CFR value comprises:
determining the ground truth value of the microvascular coronary resistance index and a value of a stenosis severity that results in FFR and CFR values that match the measured FFR value and the measured CFR value.

7. The computer-implemented method of claim 1, wherein the vessel characteristics comprise at least one of stress test assessments of the patient, coronary artery disease assessments of the patient, plaque characteristics, or perivascular adipose tissue.

8. The computer-implemented method of claim 1, wherein the patient data comprises PCCT (photon counting computed tomography) images.

9. An apparatus comprising:
means for receiving patient data of a patient, the patient data comprising patient characteristics and vessel characteristics;
means for determining an assessment of coronary microvascular disease of the patient based on the patient data using a machine learning based assessment model; and
means for outputting results of the assessment of coronary microvascular disease of the patient.

10. The apparatus of claim 9, wherein the means for determining an assessment of coronary microvascular disease of the patient based on the patient data using a machine learning based assessment model comprises:
means for determining a value of a microvascular coronary resistance index for the vessel representing a maximal reduction in coronary microvascular resistance of the vessel at maximal stress.

11. The apparatus of claim 10, wherein the machine learning based assessment model is trained using training patient data of a particular patient annotated with a ground truth value of the microvascular coronary resistance index.

12. The apparatus of claim 11, wherein the ground truth value of the microvascular coronary resistance index is determined by:
receiving a measured FFR (fractional flow reserve) value and a measured CFR (coronary flow reserve) value of a stenosis in a vessel of the particular patient;
generating a model of the vessel of the particular patient;
determining a ground truth value of a microvascular coronary resistance index using the model of the vessel based on the measured FFR value and the measured CFR value; and
outputting the ground truth value of the microvascular coronary resistance index.

13. The apparatus of claim 12, wherein the means for determining a ground truth value of a microvascular coronary resistance index using the model of the vessel based on the measured FFR value and the measured CFR value comprises:
means for determining the ground truth value of the microvascular coronary resistance index that results in FFR and CFR values that match the measured FFR value and the measured CFR value.

14. The apparatus of claim 12, wherein the means for determining a ground truth value of a microvascular coronary resistance index using the model of the vessel based on the measured FFR value and the measured CFR value comprises:
means for determining the ground truth value of the microvascular coronary resistance index and a value of a stenosis severity that results in FFR and CFR values that match the measured FFR value and the measured CFR value.

15. A non-transitory computer-readable storage medium comprising instructions which, when executed by a computer, cause the computer to carry out operations comprising:
receiving patient data of a patient, the patient data comprising patient characteristics and vessel characteristics;
determining an assessment of coronary microvascular disease of the patient based on the patient data using a machine learning based assessment model; and
outputting results of the assessment of coronary microvascular disease of the patient.

16. The non-transitory computer-readable storage medium of claim 15, wherein determining an assessment of coronary microvascular disease of the patient based on the patient data using a machine learning based assessment model comprises:
determining a value of a microvascular coronary resistance index for the vessel representing a maximal reduction in coronary microvascular resistance of the vessel at maximal stress.

17. The non-transitory computer-readable storage medium of claim 16, wherein the machine learning based assessment model is trained using training patient data of a particular patient annotated with a ground truth value of the microvascular coronary resistance index.

18. The non-transitory computer-readable storage medium of claim 17, wherein the ground truth value of the microvascular coronary resistance index is determined by:
receiving a measured FFR (fractional flow reserve) value and a measured CFR (coronary flow reserve) value of a stenosis in a vessel of the particular patient;
generating a model of the vessel of the particular patient;
determining a ground truth value of a microvascular coronary resistance index using the model of the vessel based on the measured FFR value and the measured CFR value; and
outputting the ground truth value of the microvascular coronary resistance index.

19. The non-transitory computer-readable storage medium of claim 15, wherein the vessel characteristics comprise at least one of stress test assessments of the patient, coronary artery disease assessments of the patient, plaque characteristics, or perivascular adipose tissue.

20. The non-transitory computer-readable storage medium of claim 15, wherein the patient data comprises PCCT (photon counting computed tomography) images.
